(19) United States
(12) Patent Application Publication
(10) Pub. No.: US 2023/0113847 A1
(43) Pub. Date: Apr. 13, 2023

(54) MODULATING AN IMMUNE RESPONSE WITH CUPROUS COMPLEXES

(71) Applicant: C LAB PHARMA INTERNATIONAL, S.A., Tortola (VG)

(72) Inventor: Charles Louis Albartus Barker, Flagstaff, AZ (US)

(21) Appl. No.: 17/914,320

(22) PCT Filed: Mar. 24, 2021
(86) PCT No.: PCT/IB2021/052459

§ 371 Date: Sep. 23, 2022

Related U.S. Application Data
(60) Provisional application No. 62/994,251, filed on Mar. 24, 2020.

(51) Int. Cl. A61K 31/555; A61P 21/00

(52) U.S. Cl. CPC A61K 31/555; A61P 21/00

(57) ABSTRACT

The present invention relates to methods of modulating an immune response in a subject by administering to the subject an effective amount of cuprous complex. The methods can be used to decrease the number of neutrophils and/or increase the number of ymphocytes in the subject. In certain embodiment, the methods are used to treat a subject having an inflammatory disease or conditions, for example a viral infection or chronic pain.

### CROSS REFERENCE TO RELATED APPLICATIONS

This application claims the benefit of U.S. Provisional Application No. 62/994,251, filed on Mar. 24, 2020, the contents of which are incorporated herein by reference in its entirety.

### BACKGROUND

The immune system is divided into two arms: the innate immune system and the adaptive immune system. While both are critical components in the response to pathogenic infection, they exert their activity through different cell types and in different manners. The innate immune response is mediated in part through neutrophils. Neutrophils are a type of phagocyte, and will phagocytose pathogens, but can also release a number of granules through degranulation which can aid in fighting infection and disease. Additionally, neutrophils can also form neutrophil extracellular traps, which comprise chromatin and proteases that can trap and kill pathogens. Neutrophils also play an important role in cell recruitment, and specifically in the recruitment of lymphocytes to the site of infection. The adaptive immune response is mediated largely through lymphocytes, including B cells and T cells. In the adaptive immune response, B cells mediate the humoral, or antibody-based response, whereas T cells mediate the cellular response to infection.

More recently, the ratio of neutrophils to lymphocytes has gained in popularity as a marker of infectious disease, an indicator of hyper-inflammation, and a gauge to monitor the efficiency of an immune response. The neutrophil to lymphocyte ratio is calculated by dividing the neutrophil count in a subject by the lymphocyte count and this calculation is a component of routine blood count analyses that are performed. Shifts of the neutrophil to lymphocyte ratio to favor either neutrophils or lymphocytes can alter the inflammatory response and clinical outcomes in a variety of diseases. Accordingly, new methods of modulating an immune response by altering the neutrophil to lymphocyte ratio are needed in order to develop more effective treatments in response to pathogenic infections and inflammatory diseases or conditions.

### SUMMARY

The disclosure relates to a method of modulating an immune response in a human subject. For example, the immune response is modulated by decreasing the number of neutrophils in the human subject and/or increasing the number of lymphocytes in the human subject. The method comprises administering to the human subject a composition comprising a cuprous complex and a pharmaceutically acceptable additive. In some implementations, the method further comprises determining the number of neutrophils, lymphocytes, or both in a sample from the human subject. The amount of cuprous complex administered in the composition is an amount sufficient to decrease the neutrophils in the subject to less than 4,000 per microliter of blood, increase the lymphocytes in the human subject to more than 1,300 per microliter of blood, or both. Also described are compositions for use as an immunomodulatory medicament, wherein the composition comprises a cuprous complex and a pharmaceutically acceptable additive. The cuprous complex is selected from the group consisting of: cuprous nicotinic acid, cuprous glycinate and combinations thereof. In some aspects, the cuprous complex is anhydrous. In some aspects, administering the composition to human subject comprises administering the composition twice daily.

In some implementations, the composition is administered orally, sublingually, rectally, intravenously, or epicutaneously. For examples, a composition comprising cuprous nicotinic acid is orally or sublingually administered to the subject, and a composition comprising cuprous glycinate is rectally, intravenously, or epicutaneously administered to the subject.

In some embodiments, the subject is infected with a coronavirus, for example, an Alphacoronavirus or a Betacoronavirus. In some aspects, the coronavirus is selected from the group consisting of 229E, N163, OC43, HKU1, SARS-CoV, and SARS-CoV-2. In some implementations, the subject has severe symptoms of COVID-19. In other implementations, the subject is diagnosed with chronic pain, for example, neuromuscular pain.

### BRIEF DESCRIPTION OF THE FIGURES

FIG. 1 depicts an overview of the study design.

FIGS. 2A-C depict cell counts for neutrophils (FIG. 2A), lymphocytes (FIG. 2B), and the neutrophil/lymphocyte ratio (FIG. 2C) on Day 28 in comparison to Day 0.

FIGS. 3A-C depict SQIR questionnaire results for subjects with nerve and/or muscle pain. Questionnaire scores on Day 28 in pain (FIG. 3A), the impact of the symptoms (FIG. 3B), and physical function, i.e., lifting bags of groceries (FIG. 3C) are depicted relative to scores provided at Day 0.

### DESCRIPTION OF THE INVENTION

The verb “comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there is one and only one of the elements. The indefinite article “a” or “an” thus usually means “at least one.”

The present invention is directed to copper (I) complexes (also referred to herein as “cuprous complexes”) and their use in modulating an immune response within a subject. As shown in the Examples, administration of cuprous complexes, for example anhydrous copper (I) nicotinic acid or anhydrous copper (I) glycinate (methods of production described in U.S. Pat. Nos. 9,339,506, 10,258,980, and 10,596,193), modulates an immune response. The administration of a cuprous complex alters the neutrophil to lymphocyte ratio, for example, by reducing the number of neutrophils in a subject and/or increasing the number of lymphocytes. In some aspects, the number of neutrophils per microliter of blood of a subject is decreased to less than 4,000, less than 3,500, or less than 3,250, less than 3,000, or less than 2,850. In some aspects, the number lymphocytes per microliter of blood of a subject is decreased to more than 1,300, more than 1,600, more than 1,900, more than 2,000, or more than 2,100, or more than 2,750.

In certain embodiments, the method of modulating an immune response in a human subject comprises administering to the human subject a composition comprising a cuprous complex and a pharmaceutically acceptable additive. The disclosure also relates to the use of the cuprous complex as an immunomodulatory medicament and the use of a composition comprising a cuprous complex and a pharmaceutically acceptable additive as an immunomodulatory medicament. In certain implementations, the cuprous complex is copper (I) nicotinic acid or is copper (I) glycinate. The pharmaceutically acceptable additive includes pharmaceutically acceptable carriers, adjuvants, and vehicles. Preferably, the pharmaceutically acceptable carrier is an inert diluent. In some aspects, the cuprous complex in the composition is anhydrous.

In certain implementations, the method further comprises determining the number of neutrophils, lymphocytes, or both in a blood sample from the human subject. In some aspects, the amount of cuprous complex administered in the composition is an amount sufficient decrease the neutrophils in the subject to less than 4,000 cells per microliter of blood, for example, less than 3,500, or less than 3,250, less than 3,000, or less than 2,850. In some aspects, the amount of cuprous complex administered in the composition is an amount sufficient increase the neutrophils in the subject to more than 1,300 cells per microliter of blood, for example, more than 1,300, more than 1,600, more than 1,900, more than 2,000, or more than 2,100, or more than 2,750. In particular embodiments, the amount of cuprous complex administered in the composition is 5-15 mg, for example, between 5 mg and 13 mg, between 6 mg and 13 mg, 12±0.5 mg, or 6±0.5 mg.

The cuprous complex and the composition comprising the cuprous complex can be administered orally, sublingually, rectally, intravenously, or epicutaneously. For examples, a composition comprising cuprous nicotinic acid is orally or sublingually administered to the subject, and a composition comprising cuprous glycinate is rectally, intravenously, or epicutaneously administered to the subject. In some implementations, the cuprous complex and the composition comprising the cuprous complex are administered once or twice daily. Thus, in some aspects, the effective amount of the cuprous complex and the composition comprising the cuprous complex may be split into two doses that are administered daily.

Copper (I) nicotinic acid has an acidic pH. Accordingly, this cuprous complex is more suited to oral administration rather than intravenous or rectal administration. Copper (I) glycinate has a more neutral pH. Thus, this cuprous complex suited for intravenous, rectal, or oral administration. In fact, the anhydrous copper (I) glycinate complex prepared according to the methods described U.S. Pat. No. 10,596,193 can also be administered epicutaneously, where the complex is rubbed or massaged into the skin of the human subject.

The described method and medicaments are useful in the treatment of various diseases involving inflammation and/or chronic inflammation, in particular, diseases where patients exhibit higher neutrophil counts, lower lymphocyte counts, and ultimately a higher neutrophil to lymphocyte ratio. Thus, the diseases and conditions treatable by the described methods and medicament include, but not limited to, coronavirus infection, influenza virus infection, coronary heart diseases, pancreatitis, chronic inflammatory diseases including ulcerative colitis and rheumatoid arthritis, adult-onset Still's disease, and multiple sclerosis.

For example, in coronary heart disease, a higher neutrophil to lymphocyte ratio has been associated with a worse prognosis in patients with various forms of coronary heart disease. Specifically, a study by Kim et al. found that a higher neutrophil to lymphocyte ratio is associated with coronary heart disease mortality, left atrial thrombus, and myocardial perfusion (Kim S, et al., “Association of Neutrophil-to-Lymphocyte Ratio with Mortality and Cardiovascular Disease in the Jackson Heart Study and Modification by the Duffy Antigen Variant.” JAMA Cardiol., 2018, 3(6):455-462).

As the neutrophil to lymphocyte ratio is a marker for inflammation and an inflammatory response, the neutrophil to lymphocyte ratio is particularly pertinent to lung infections and diseases causing an inflammatory response. Persistent lung inflammation, and particularly neutrophilic inflammation exacerbates disease and leads to adverse clinical outcomes. A study by Zhang et al. indicated that the neutrophil to lymphocyte ratio was an effective predictor of disease severity in patients infected with avian influenza virus type H7N9 (AIV-H7N9) (Zhang Y, et al., “Neutrophil-Lymphocyte ratio as an early new marker in AIV-H7N9-infected patients: a retrospective study.” Ther Clin Risk Manag. 2019, 15:911-919). Zhang et al. found that higher neutrophil to lymphocyte ratio was associated with more severe disease and increased fatality rates.

Coronaviruses (CoV), and particularly severe acute respiratory syndrome coronavirus (SARS-CoV), Middle East respiratory syndrome coronavirus (MERS-CoV), and severe acute respiratory syndrome coronavirus 2 (SARS-CoV-2) cause severe respiratory infections. To date, no specific antiviral treatments exist for treating these CoVs and individuals can only receive medical care that relieves symptoms or in severe cases, support vital organ functions. A comparison of blood samples from patients suffering from severe and non-severe symptoms of COVID-19 hospitalized in Tonji Hospital (Wuhan, China) from Jan. 10, 2020 to Feb. 12, 2020 showed that patients with severe symptoms have increased number of neutrophils and decreased numbers of lymphocytes when compared to patient with non-severe symptoms. Most COVID-19 patients experienced symptoms of fever, shortness of breath, expectoration, fatigue, dry cough, and myalgia. Patient with severe symptoms were those also experiencing respiratory distress with the respiratory rate over 30 per minute, having oxygen saturation of less than or equal to 93% at resting state, and having the arterial blood oxygen partial pressure (PaO₂)/oxygen concentration (FiO₂) be less than or equal to 300 mm Hg. Based on these blood test results, it was suggested that patients suffering from severe symptoms of COVID-19 have an out-of-control innate immune response to SARS-CoV-2. Accordingly, administration of a cuprous complex is useful in the reduction of the likelihood of developing severe symptoms of COVID-19 and in the reduction the severity of symptoms associated with COVID-19 by decreasing the number of neutrophils and increasing the number of lymphocytes in a subject.

While SARS-CoV, MERS-CoV, and SARS-CoV-2 result in significant and severe respiratory disease and inflammation, other CoVs also cause respiratory and gastrointestinal infections where there is an inflammatory response. These CoVs include the Alphacoronaviruses HCoV-NL63 and HCoV-229E, and the Betacoronaviruses HCoV-0C43, HCoV-HKU1, HCoV-NL63 and HCoV-229E. SARS-CoV, MERS-CoV, and SARS-CoV-2 are also Betacoronavirus. HCoV-NL63, HCoV-229E, HCoV-OC43, and HCoV-HKU1, for example, are pathogens that cause the common cold and stimulate an inflammatory response. Modulation of the neutrophil to lymphocyte ratio is important in mediating an effective immune response and the present invention therefore is useful in the treatment of CoV infections by decreasing the number of neutrophils and increasing the number of lymphocytes in a subject.

Interestingly, administration of anhydrous copper (I) nicotinic acid to otherwise healthy subjects experiencing chronic pain also resulted in a significant decrease in the number neutrophils along with a significant increase in the number of lymphocytes. Emerging clinical results have shown that modifying the neutrophil to lymphocyte ratio by decrease in the number neutrophils along with a significant increase in the number of lymphocytes is beneficial in promoting resolution of chronic pain. Reports of reduced pain coincided with the altered neutrophil to lymphocyte ratio in otherwise healthy subjects experiencing chronic pain. Because many cases of neuromuscular pain and chronic pain stem from inflammation, control of the inflammatory response directly impacts pain and other symptoms that a subject may experience. Reducing the neutrophil to lymphocyte ratio reduces inflammation and the side-effects that come from neutrophilic inflammation. Thus, the disclosed methods and medicaments are also suited for the treatment of chronic inflammatory diseases and/or for reducing pain in subjects with chronic nerve and/or muscle pain.

The present invention is further illustrated by the following examples that should not be construed as limiting.

### EXAMPLES

### 1. Materials and Methods

Ethics: This study was reviewed by the Natural Health Products Directorate (NHPD), Health Canada and a research ethics board. This study was conducted in accordance with the principles established in the Declaration of Helsinki and its subsequent amendments. Informed consent was obtained from each subject at the screening visit prior to any study related activities. The clinical trial was conducted at KGK Synergize Inc. (London, ON, Canada).

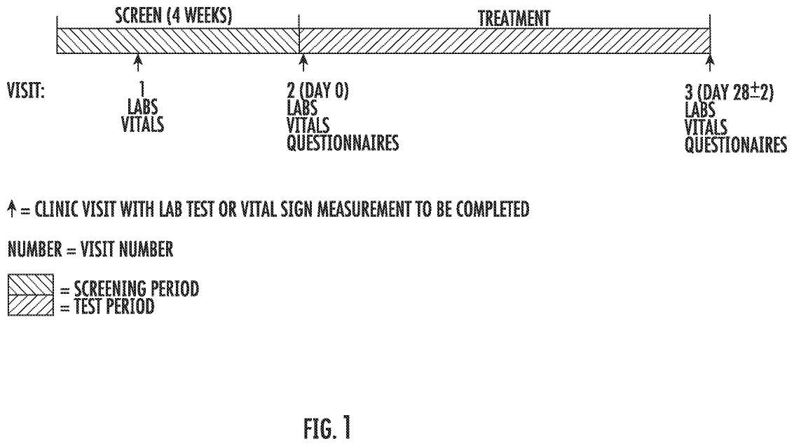

Sample Collection: As depicted in FIG. 1, blood was collected from participants on the initial screening (Day −1), the first day of administering the treatment composition (Day 0), and on Day 28. Collected blood was analyzed to determine the complete blood count (CBC), electrolyte composition, and liver enzymes composition according to standardized procedures at LifeLabs (London, ON, Canada).

Treatment Composition: Active Ingredients (per capsule): Cunermuspir (Copper Niacin Chelate) 6.06 mg; Other ingredients: Organic evaporated cane juice powder, hypromellose, titanium dioxide.

Placebo Composition: Organic evaporated cane juice powder, hypromellose, titanium dioxide.

Dosing Regimen: Each participant was randomly assigned an identifier code and placed in either the Cunermuspir or placebo treatment groups at a 1:1 ratio. Participants were given 2 capsules on Day 0 and instructed to take the contents sublingually with a small meal. Starting on Day 1, participants were instructed to take one capsule orally in the morning with breakfast, and one capsule orally between lunch and dinner, with food, for a total of 2 capsules daily over the course of the study. Participants were instructed to take all capsules with adequate, but not excessive, amounts of liquid. Capsules were taken with food to reduce the potential for side effects. If a subject missed a dose, they were instructed to take the next dose as they remembered. Participants were instructed not to exceed 2 capsules daily. Participants were also asked to consume a minimum of six 8 oz glasses of water daily during the study. Any participants on prescription or over the counter medications were asked to take their medications at least two hours before or after taking the treatment. Compliance with the treatment regimen was assessed by counting the returned unused test product at each visit. Participants found to have a compliance of <80% or >120% at any visit were counseled. A compliance of <70% or >130% was considered as non-compliant and any subject demonstrating non-compliance for two consecutive visits was withdrawn from the study and not included in the statistical analysis.

Blinding: The investigational product bottles were labeled according to the requirements of ICH-GCP guidelines and applicable local regulatory guidelines. Investigational product was coded by the unblended personnel at KGK Synergize who were not involved in the study assessments. Further, each participant was assigned a randomized code.

Questionnaires: Study participants were asked to complete questionnaires to assess the effect of Cunermuspir complex on quality of life. Quality of life was assessed with the Individualized Neuromuscular Quality of Life Questionnaire (INQoL). Changes from baseline in physical function, pain, and fatigue/energy were assessed by the Symptom Impact Questionnaire (SQIR) and INQoL. The impact of Cunermuspir on cognitive function was assessed by Mini-Mental State Examination (MMSE).

Statistics: Unless otherwise noted, statistical analyses were performed using R, and a P value of <0.05 was considered statistically significant. Comparisons of log-transformed data between the treatment and placebo groups were done using Analysis of Covariance (ANCOVA) and Two-way Analysis of Variance (ANOVA), and analyses within a group were performed by paired Student t-test. For data that was not log-transformed, analyses were performed by non-parametric Mann Whitney U test for comparisons between treatment groups, and by signed-ranks test for analyses performed within a group.

### 2. Cuprous Nicotinic Acid Decreases the Number of Neutrophils and Increases the Number of Lymphocytes in Human Subjects.

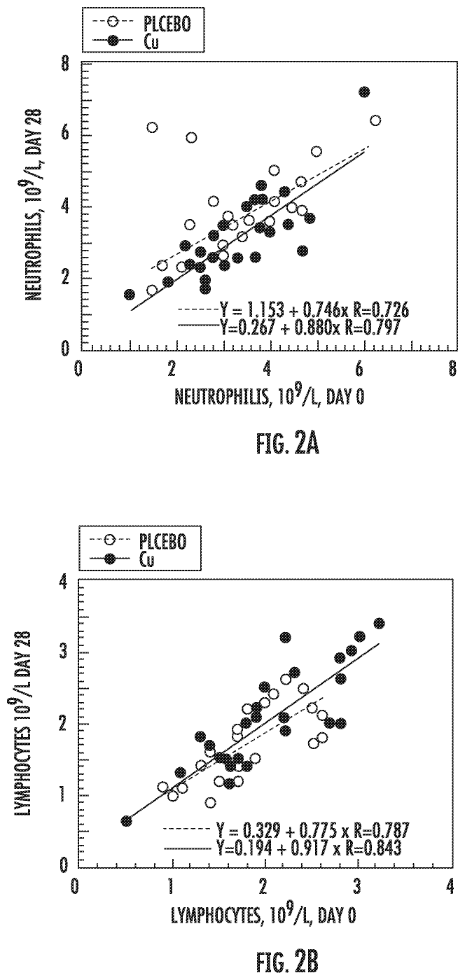

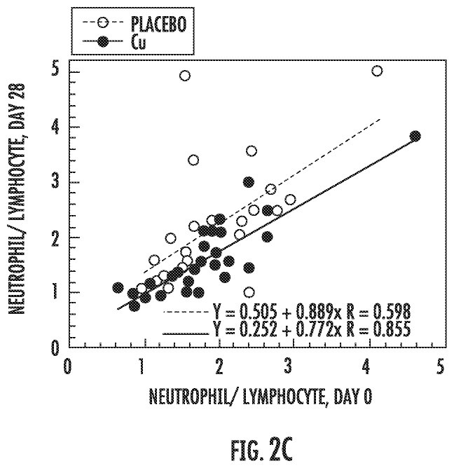

Table 1 depicts the number of neutrophils, lymphocytes, and the calculated neutrophil to lymphocyte ratio in subjects that received the placebo or Cunermuspir (Cu(I)NA₂) over the 28-day study period. At the initial sampling taken at Day −1 (Screening) and Day 0 (Baseline), there was no significant difference in number of neutrophils, lymphocytes, or in the neutrophil to lymphocyte ratio in participants in placebo and Cunermuspir groups. However, by Day 28, after twice daily administration of Cunermuspir, participants in the Cunermuspir group exhibited a significant decrease in neutrophil numbers, a significant increase in lymphocyte numbers, and a significant decrease in the neutrophil to lymphocyte ratio relative to the placebo group (FIGS. 2A-2C). Notably, two subjects in the placebo group exhibited a neutrophil to lymphocyte ratio of greater than 4.0, whereas no subject that was administered Cunermuspir had a neutrophil to lymphocyte ratio above 4.0 at Day 28 (FIG. 2C). Together, these data indicate that Cunermuspir modulates an immune response by altering the neutrophil to lymphocyte ratio, which impacts the effectiveness of an immune response against pathogenic infection.

TABLE 1
Effect of Cunermuspir on the number of Neutrophils, Lymphocytes,
and the Neutrophil to Lymphocyte ratio in subjects.

| | | |
|---|---|---|
| Placebo | Cu(I)NA₂ | |
| Mean ± SD (N) | Mean ± SD (N) | |
| Median (Min-Max) | Median (Min-Max) | P Value |

Neutrophils (10⁹/L)

| Screening | 3.49 ± 1.12 | (28) | 3.45 ± 1.16 | | — |
|---|---|---|---|---|---|
| | 3.4 | (1.8-6.4) | 3.2 | (1.3-6.3) | |
| Baseline | 3.35 ± 1.13 | (28) | 3.28 ± 1.06 | (28) | — |
| | 3.1 | (1.5-6.2) | 3.15 | (1.3-6.3) | |
| Visit 3 (Day 28) | 3.60 ± 1.13 | (27) | 3.15 ± 1.19 | (26) | 0.03 |
| | 3.6 | (1.7-6.4) | 2.85 | (1.5-7.2) | |
| Change from | 0.32 ± 0.83 | (27) | −0.13 ± 0.73 | (26) | — |
| Baseline to Visit 3 | 0.2 | (−0.8-3.6) | 0.05 | (−1.9-1.2) | |

| | |
|---|---|
| p = 0.05 | p = 0.38 |

Lymphocytes (10⁹/L)

| Screening | 1.84 ± 0.51 | (28) | 2.15 ± 0.69 | | — |
|---|---|---|---|---|---|
| | 1.9 | (0.8-2.8) | 2.15 | (0.6-3.4) | |
| Baseline | 1.83 ± 0.51 | (28) | 2.01 ± 0.64 | (28) | — |
| | 1.8 | (0.9-2.7) | 1.9 | (0.5-3.2) | |
| Visit 3 (Day 28) | 1.74 ± 0.51 | (27) | 2.04 ± 0.72 | (26) | <0.01 |
| | 1.8 | (0.9-2.6) | 2.0 | (0.6-3.4) | |
| Change from | −0.08 ± 0.34 | (27) | 0.03 ± 0.72 | (26) | — |
| Baseline to Visit 3 | 0 | (−0.8-0.4) | 0.1 | (−0.8-1.0) | |

| | |
|---|---|
| p = 0.22 | p = 0.73 |

Neutrophil to Lymphocyte Ratio

| Screening | 1.90 ± 0.13 | | 1.77 ± 0.15 | | |
|---|---|---|---|---|---|
| | 1.76 | (1.14-3.72) | 1.67 | (0.62-4.57) | |
| Baseline | 1.90 ± 0.68 | | 1.77 ± 0.77 | | p = 0.44 |
| | 1.75 | (0.96-4.1) | 1.75 | (0.625-4.6) | |
| Visit 3 (Day 28) | 2.20 ± 1.03 | | 1.62 ± 0.69 | | p = 0.07 |
| | 2.08 | (1.0-5.0) | 1.44 | (0.77-3.83) | p < 0.05 |
| | p = 0.066 | | p = 0.054 | | p < 0.01 |
| Change from | −0.294 ± 0.813 | | 0.152 ± 0.40 | | p < 0.05 |
| Baseline to Visit 3 | −0.049 | (−3.38-1.38) | 0.123 | (−0.625-0.902) | |

3. Cuprous Nicotinic Acid Reduces Pain in Subjects with Chronic Neuromuscular Pain.

Cuprous nicotinic acid (Cunermuspir) was further investigated for its ability to provide relief for subjects experiencing neuromuscular pain, including fibromyalgia neuromuscular pain. Participants in the above-described study were assessed over a 28-day period to evaluate the effect that Cunermuspir has on physical functionality, pain, and energy/fatigue.

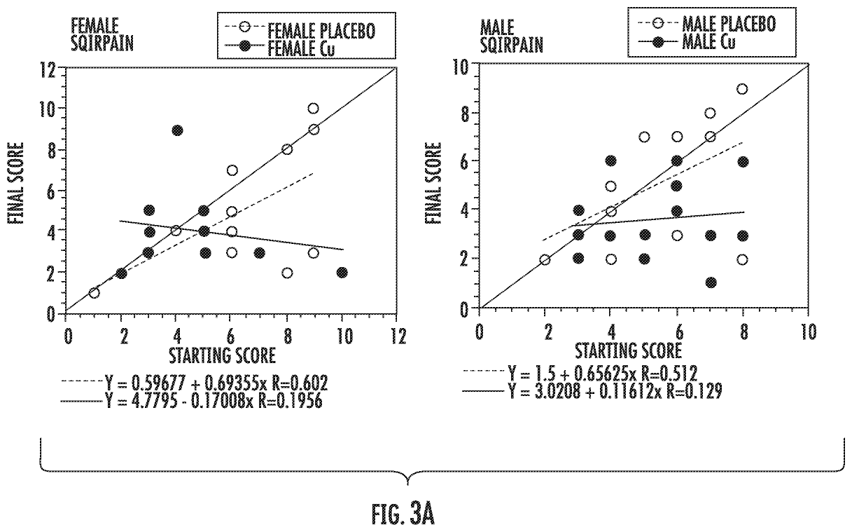

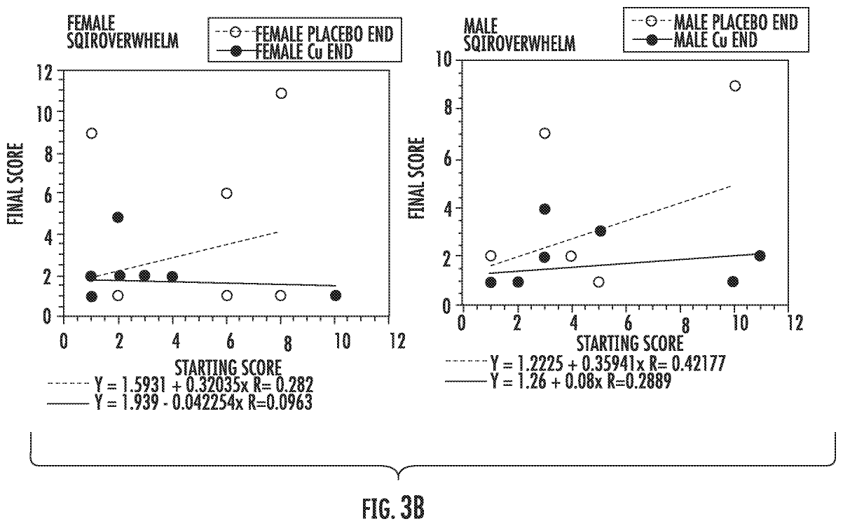

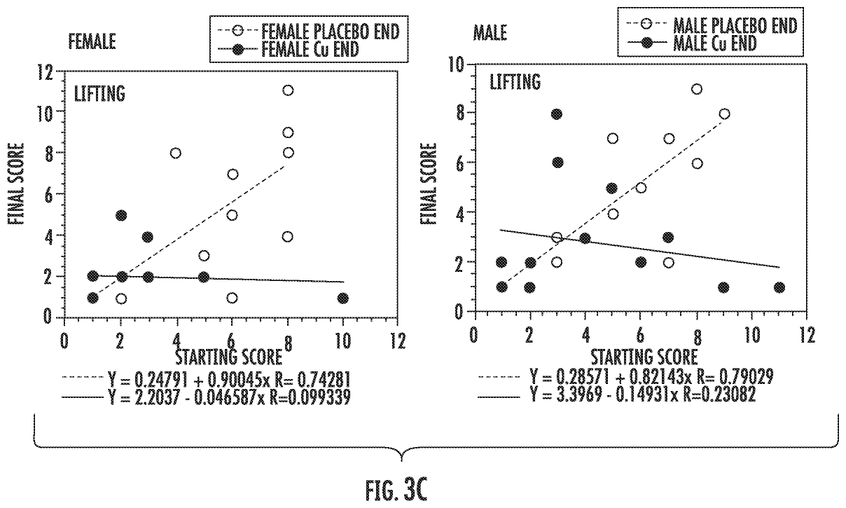

It was found that twice daily administration of Cunermuspir over the 28-day trial period was associated with decreased scores in subjects with neuromuscular pain at Day 28 in comparison to Day 0 in the Symptom, Impact, and Function Domains that are associated with the SQIR questions (FIGS. 3A-3C). Notably, subjects taking Cunermuspir experienced a downward trend in their final scoring relative to their starting score. Conversely, subjects in the placebo group exhibited an upward trend in final scores relative to starting scores in all SQIR categories measured (FIGS. 3A-3C), indicating that Cunermuspir helped alleviate symptoms associated with neuromuscular pain. These improvements in SQIR scoring are generally associated with an improvement in quality of life and indicate that Cunermuspir is associated with an overall reduction in pain and improvements in physical function.

What is claimed is:

1. A method of modulating an immune response in a human subject, the method comprising administering to the human subject a composition comprising a cuprous complex selected from the group consisting of: cuprous nicotinic acid, cuprous glycinate and combinations thereof; and a pharmaceutically acceptable additive.

2. The method of claim 1, further comprising determining the number of neutrophils, lymphocytes, or both in a blood sample from the human subject.

3. The method of claim 1 or 2, wherein modulating the immune system includes decreasing the number of neutrophils in the human subject and/or increasing the number of lymphocytes in the human subject.

4. The method of claim 2 or 3, wherein the cuprous complex is administered in an amount sufficient to decrease the neutrophils in the human subject to less than 4,000 per microliter of blood, and/or to increase the lymphocytes in the human subject to more than 1300 per microliter of blood, or both.

5. The method of any one of claims 1-4, wherein the composition is administered twice daily and the cuprous complex is anhydrous.

6. The method of any one of claims 1-5, wherein the composition comprises cuprous nicotinic acid, the composition is orally administered to the human subject.

7. The method of any one of claims 1-5, wherein the composition comprises cuprous nicotinic acid, the composition is sublingually administered to the human subject.

8. The method of any one of claims 1-5, wherein the composition is cuprous glycinate, the composition is rectally administered to the human subject.

9. The method of any one of claims 1-5, wherein the composition is cuprous glycinate, the composition is intravenously administered to the human subject.

10. The method of any one of claims 1-5, wherein the composition is cuprous glycinate, the composition is epicutaneously administered to the human subject.

11. The method of any one of claims 1-10, wherein the subject is infected with a coronavirus.

12. The method of claim 11, wherein the coronavirus is SARS-CoV or SARS-CoV-2.

13. The method of any one of claims 1-12, wherein the human subject is a patient with severe symptoms of COVID-19.

14. The method of any one of claims 1-11, wherein the subject is diagnosed with chronic pain.

15. The method of claim 14, wherein the chronic pain is neuromuscular pain.

16. A composition for use as an immunomodulatory medicament, the composition comprising:
a cuprous complex selected from the group consisting of: cuprous nicotinic acid, cuprous glycinate and combinations thereof; and
a pharmaceutically acceptable additive.

17. A composition for use to decrease the number of neutrophils in a human subject and/or to increase the number of lymphocytes in a human subject, the composition comprising:
a cuprous complex selected from the group consisting of: cuprous nicotinic acid, cuprous glycinate and combinations thereof; and
a pharmaceutically acceptable additive.

18. The composition for the use of claim 16 or 17, wherein the cuprous complex is anhydrous.

19. The composition for the use of any one of claims 16-18, wherein composition is orally administered, the cuprous complex is cuprous nicotinic acid.

20. The composition for the use of any one of claims 16-19, wherein the composition is sublingually administered, the cuprous complex is cuprous nicotinic acid.

21. The composition for the use of any one of claims 16-18, wherein the composition is rectally administered, the cuprous complex is cuprous glycinate.

22. The composition for the use of any one of claims 16-18, wherein the composition is intravenously administered, the cuprous complex is cuprous glycinate.

23. The composition for use of any one of claims 16-18, wherein the composition is epicutaneously administered, the cuprous complex is cuprous glycinate.

24. The composition for the use of any one of claims 16-23, wherein the composition is administered twice daily.

25. The composition for the use of any one of claims 16-24, wherein the human subject is infected with a coronavirus.

26. The composition for the use of claim 25, wherein the coronavirus is SARS-CoV or SARS-CoV-2.

27. The composition for the use of any one of claims 16-26, wherein the human subject is a patient with severe symptoms of COVID-19.

28. The composition for the use of any one of claims 16-24, wherein the human subject is diagnosed with chronic pain.

29. The composition for the use of claim 28, wherein the chronic pain is neuromuscular pain.
